(19) United States
(12) Patent Application Publication
(10) Pub. No.: US 2026/0354317 A1
(43) Pub. Date: Nov. 12, 2026

(54) PRODUCTION OF A PSYCHOACTIVE ALKALOID AND COMPOSITIONS, AND PRODUCTION OF A FRUITING BODY ORGANISM

(71) Applicant: FUTURE WELLNESS LIMITED, Kingston 10, Saint Andrew (JM)

(72) Inventors: Rupika Delgoda, Kingston 7 (JM); Kimberley Foster, Kingston 7 (JM); Isaac Morrison, Kingston 6 (JM); Marshall Tyler, Monterey, CA (US)

(21) Appl. No.: 19/139,694

(22) PCT Filed: Dec. 14, 2023
(86) PCT No.: PCT/IB2023/062688

§ 371 Date: Jun. 16, 2025

Related U.S. Application Data
(60) Provisional application No. 63/433,296, filed on Dec. 16, 2022.

(51) Int. Cl. A01G 18/40; A01G 18/20; C07D 209/16; C07F 9/572

(52) U.S. Cl. CPC A01G 18/40; A01G 18/20; C07D 209/16; C07F 9/5728

(57) ABSTRACT

There is disclosed a process for the production of a psychotropically active compound. The process comprises the step of: (a) culturing fungal material of Psilocybe subtropicalis in a growth medium to produce a composition comprising the psychotropically active compound, and (b) isolating the composition from the growth medium. Preferably, the process used to produce a novel mycelial mat which is another aspect of the invention. There is also disclosed a novel growth medium preferably useful for culturing fungal material. There is also disclosed a process for the production of an intact mushroom fruiting organism and the intact mushroom fruiting organism, per se.

### CROSS-REFERENCE TO RELATED APPLICATION

The present application claims the benefit under 35 U.S.C. § 119(e) of provisional patent application Ser. No. 63/433,296, filed Dec. 16, 2022, the contents of which are hereby incorporated by reference.

### BACKGROUND OF THE INVENTION

### Field of the Invention

The present invention generally relates to processes for production of a psychoactive alkaloid and production of a fruiting body organism. In one preferred aspect, the present invention relates to a process for production of a psychoactive alkaloid (e.g., psilocybin and/or psilocin), more preferably in the form of a mycelial mat. More specifically and preferably, this aspect of the present invention relates to culturing fungal material of Psilocybe subtropicalis to produce a psychotropically active compound. In another preferred aspect, the present invention relates to a process for production of a fruiting body organism, preferably an intact fruiting body organism of a mushroom such as Psilocybe subtropicalis. In another preferred aspect, the present invention relates to liquid growth medium. Other aspects of the present invention will become apparent to those of skill in the art upon review of this specification.

### Description of the Prior Art

Varieties of mushrooms have played important roles in most societies. The active ingredients in mushrooms, especially psilocybin mushrooms with psychoactive compounds such as psilocybin and psilocin, as well as related molecules such as baeocystin, norbaeocystin, and norpsilocin, have been found to have medicinal properties including relief of symptoms of various diseases and conditions. The concentration of active psilocybin mushroom compounds varies not only from species to species, but also from mushroom to mushroom inside a given species, subspecies or variety. The same holds true even for different parts of the same mushroom.

Various methods of extraction, which have been used to separate natural extracts from a variety of mushrooms, have resulted in difficulties with large crop-to-crop variability. There is as well the problem of a large variability within a single fungus in terms of the concentration of the active psychoactive compound and its stability. Different solvent choices extract the psychoactive compounds variably, some of them selectively extract one or the other, and some convert the compounds between each other or degrade them into non-psychoactive compounds. Many extraction processes for extracting standardized concentrations of the compounds for direct medical use are usually complex. This results in expensive extraction processes and a high cost of isolated, natural extracts.

U.S. Pat. No. 3,183,172 [Heim et al.] relates to an industrial process for the isolation of active compounds from mushrooms grown under predetermined conditions. With the predetermined growing conditions, mushrooms grow with ten times more active mycelium and sclerotium, and increased concentrations of psychoactive compounds. However, a large portion of the target compounds are lost during the extraction process or not extracted at all. In addition, the solvent and solvent systems used during the extraction process are materials such as methanol, acetone, dichloromethane, diethyl ether or others known to be toxic to humans, even in small quantities. These problems are significant with respect to very potent extracts of psilocybin mushrooms, considering that a normal dose for use ranges from only 5 mg to 25 mg. The extracted psychoactive compounds are generally without a stable and standardized concentration.

To date, the focus has largely been on synthetic preparations of these compounds because of the many difficulties associated with naturally extracted preparations. It is currently infeasible and expensive to extract psilocybin from mushrooms on a large/commercial scale, and even the best chemical synthesis methods require expensive and difficult-to-source starting substrates.

Accordingly, there is a need in the art for an improved process for producing a psychotropically active compound from fungal material of a naturally occurring mushroom in a high yield which obviates or mediates at least one of the above disadvantages of the prior art.

In addition, processes to grow fruiting bodies such as mushrooms are generally known. However, these known processes have some disadvantages associated with them. For example, these known processes generally rely on growth of the mushroom fruiting body in a solid substrate such as soil. When grown in such a manner, it is very difficult to grow and harvest an intact mushroom fruiting body (particularly in a reproducible fashion)—i.e., a fruiting body organism containing all components such as caps, gills, stems, hyphae, and more particularly, mycelium. In addition, growth in a solid substrate such as soil results in a production of a non-sterile, mushroom fruiting body which must be further processed to produce a relatively cleaner product. This further processing results in additional cost in the overall process and/or can result in loss of valuable components in the mushroom fruiting body organism.

Accordingly, there is a need in the art for an improved process for production of a mushroom fruiting organism, preferably an intact fruiting body organism of a mushroom such as Psilocybe subtropicalis which obviates or mediates at least one of the above disadvantages of the prior art.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

It is an object of the present invention to obviate or mitigate at least one of the above-mentioned disadvantages of the prior art.

It is another object of the present invention to provide a novel process for producing a psychotropically active compound from fungal material of a naturally occurring mushroom.

Accordingly, in one of its aspects, the present invention provides a process for the production of a psychotropically active compound comprising the steps of: (a) culturing fungal material of Psilocybe subtropicalis in a growth medium to produce a composition comprising the psychotropically active compound, and (b) isolating the composition from the growth medium.

In another of its aspects, the present invention provides a mycelial mat produced by harvesting fungal material of Psilocybe subtropicalis in a growth composition.

A liquid growth medium composition comprising: a yeast extract, a malt extract, dextrose, tryptamine and, optionally, agar.

In another of its aspects, the present invention provides a composition comprising mycelium of Psilocybe subtropicalis and a psychotropically active compound, wherein the psychotropically active compound is present in an amount of at least about 5 mg/g dried composition.

In another of its aspects, the present invention relates to a process for the production of an intact mushroom fruiting organism, the process comprising the step of culturing fungal material of a previously harvested mushroom fruiting body in a liquid growth medium in a vessel.

In another of its aspects, the present invention relates to an intact mushroom fruiting organism in substantially pure form.

For one aspect of the invention, the present inventors have surprisingly and unexpectedly discovered that the selection of Psilocybe subtropicalis as the source fungal material surprisingly results in significantly higher yields of psychotropically active compound (e.g., psilocybin and/or psilocin) on a dry basis compared to when other psilocybin-producing fungal materials, such as Psilocybe cubensis, Panaeolus cyanescens, and Gymnopilus sp., are chosen as the source fungal material. It is highly preferred to use the mycelia of Psilocybe subtropicalis as the source fungal material. The present inventors have discovered that process of this aspect of the invention can be used to produce a mycelial mat wherein the psychotropically active compound is present in an amount of at least about 5 mg/g dried mat composition, more preferably at least about 8 mg/g dried mat composition, more preferably in the range of from about 8 to about 20 mg/g dried mat composition, more preferably from about 10 to about 20 mg/g dried mat composition, 12 to about 18 mg/g dried mat composition—this is believed to be a novel composition of matter. In addition, the present inventors have surprisingly and unexpectedly discovered that these significantly higher yields can be achieved in a harvest period that is a matter of weeks long—i.e., much more quickly than known cultivation cycles (i.e., growth and harvest) which typically take 2-3 months. In addition, the present inventors have discovered that, conducting step (a) [i.e., the culturing step above] in the presence of tryptamine results in surprisingly increased concentration of the desired psychotropically active compound. In this regard, the present inventors have discovered that, despite attempts to grow other species in similar ways, Psilocybe subtropicalis surprsingly and unexpectedly appears to be unique in its tolerance to high tryptamine concentrations and in its ability to produce such potent mycelial mats. For example, the biological efficiency of producing P. subtropicalis mat this way is 121% in comparison to 45% for P. cubensis mat, 70% each for P. cyanescens and Gymnopilus sp.

For another aspect of the invention, the present inventors have discovered a process for the production of an intact mushroom fruiting organism. The process generally involves harvesting fungal material of the mushroom fruiting organism in a liquid with or without agar, in a vessel. In a preferred embodiment, the vessel is configured to (i) permit air circulation between the interior and exterior of the vessel, and/or (ii) exclude ingress of microorganisms exterior to the vessel. In a preferred embodiment, the mushroom fruiting organism is harvested from a liquid growth medium comprising inner cap tissue from a previously harvested mushroom fruiting body. In a highly preferred embodiment, the process is related to production of an intact mushroom fruiting organism of Psilocybe subtropicalis although the present inventors believe the process can be applied to other mushroom species. The present inventors have discovered that this process can be used to produce an intact mushroom fruiting organism at significantly shortened incubation periods (e.g., as little as 3 weeks) and using fewer steps compared to known harvesting processes from a solid substrate (these typically take 2-3 months). Moreover, such a process circumvents multiple of the normally requisite steps in the cultivation process (e.g., inoculation of grain and spawning to bulk substrate).

### BRIEF DESCRIPTION OF THE DRAWINGS

Embodiments of the present invention will be described with reference to the accompanying drawing, in which:

FIGS. 1-2 illustrate the results obtained in Example 1 reported below;

FIGS. 3-4 illustrate the results obtained in Example 2 reported below.

### DETAILED DESCRIPTION OF THE PREFERRED EMBODIMENTS

In one of its aspects, the present invention relates to a process for the production of a psychotropically active compound comprising the steps of: (a) culturing fungal material of Psilocybe subtropicalis in a growth medium to produce a composition comprising the psychotropically active compound, and (b) isolating the composition from the growth medium. Preferred embodiments of this process may include any one or a combination of any two or more of any of the following features:
the psychotropically active compound is psilocybin;
the psychotropically active compound is psilocin;
the psychotropically active compound is a mixture comprising psilocybin and psilocin;
the growth medium further comprises tryptamine;
tryptamine is present at a concentration of greater than about 0.3 mM;
tryptamine is present at a concentration in the range of from about 0.3 mM to about 2.0 mM;
tryptamine is present at a concentration in the range of from about 0.6 mM to about 1.5 mM;
tryptamine is present at a concentration of about 1.2 mM;
the step of culturing is conducted at a temperature in the range of from about 15 °C to about 30 °C;
the step of culturing is conducted at a temperature in the range of from about 20 °C to about 25 °C;
the step of culturing is conducted at a temperature in the range of from about 22 °C to about 25 °C;
the step of culturing is conducted in the presence of a growth medium;
the growth medium comprises a carbohydrate and a yeast component;
the carbohydrate comprises dextrose;
the yeast component comprises a yeast malt broth;
the step of culturing is conducted in cycles of darkness and light exposure;
the cycles of darkness and light exposure are of substantially the same duration;
the duration is from about 2 hours to about 24 hours;
the duration is from about 6 hours to about 18 hours;
the duration is from about 10 hours to about 14 hours;
the duration is about 12 hours;
the step of culturing is conducted for a period of at least 1 day;
the step of culturing is conducted for a period of at least 1 week;
the step of culturing is conducted for a period of from 1 week to 12 weeks;
the step of culturing is conducted for a period of from 2 week to 4 weeks;
Step (a) is conducted for a period of about 3 weeks; and/or
the fungal material is the mycelia of Psilocybe subtropicalis and the composition produced in Step (b) is a mycelial mat.

This aspect of the invention also relates to an isolated and dried mat produced according to the above process and any of its preferred embodiments.

In another of its aspects, the present invention relates to a liquid growth medium composition comprising: a yeast extract, a malt extract, dextrose, tryptamine and, optionally, agar. In a preferred embodiment, the liquid growth medium is in the form of an aqueous liquid growth medium (that may or may not contain agar). In a highly preferred embodiment, the growth medium is in the form of an aqueous solid growth medium. Preferred embodiments of this medium (aqueous or non-aqueous) may include any one or a combination of any two or more of any of the following features:
the yeast extract is present at a concentration in the range of from about 1 g/L to 5 g/L;
the yeast extract is present at a concentration in the range of from about 2 g/L to 4 g/L;
the yeast extract is present at a concentration of about 3 g/L;
the malt extract is present at a concentration in the range of from about 1 g/L to 5 g/L;
the malt extract is present at a concentration in the range of from about 2 g/L to 4 g/L;
the malt extract is present at a concentration of about 3 g/L;
the growth medium composition further comprises peptone;
peptone is present at a concentration in the range of from about 3 g/L to 7 g/L;
peptone is present at a concentration in the range of from about 4 g/L to 6 g/L;
peptone is present at a concentration of about 5 g/L;
dextrose is present at a concentration in the range of from about 8 g/L to 12 g/L;
dextrose is present at a concentration in the range of from about 9 g/L to 11 g/L;
dextrose is present at a concentration of about 10 g/L;
tryptamine is present at a concentration in the range of from about 0.3 mM to about 2.0 mM;
tryptamine is present at a concentration in the range of from about 0.6 mM to about 1.5 mM;
tryptamine is present at a concentration of about 1.2 mM;
the growth medium composition further comprises agar;
agar is present at a concentration in the range of from about 0 g/L to 30 g/L;
agar is present at a concentration in the range of about 3 g/L to about 15 g/L; and/or
agar is present at a concentration of about 10 g/L.

In another of its aspects, the present invention relates to a process for the production of an intact mushroom fruiting organism, the process comprising the step of culturing fungal material of a previously harvested mushroom fruiting body in a liquid growth medium in a vessel. Preferred embodiments of this process may include any one or a combination of any two or more of any of the following features:
the fungal material comprises inner cap tissue from the previously harvested mushroom fruiting body;
the previously harvested mushroom fruiting body belongs to the species Psilocybe subtropicalis;
the step of culturing is conducted for up to 8 weeks;
the step of culturing is conducted for up to 6 weeks;
the step of culturing is conducted for 3-5 weeks;
the step of culturing is conducted for about 4 weeks;
the vessel is configured to (i) permit air circulation between the interior and exterior of the vessel, and/or (ii) exclude ingress of microorganisms exterior to the vessel, and/or (iii) be made of a material which is radiation transparent (e.g., transparent to ambient radiation);
the vessel is made of a radiation transparent (e.g., glass);
the liquid growth medium comprises the liquid growth medium composition disclosed in Paragraph [0024];
the step of culturing is conducted in cycles of darkness and light exposure;
the cycles of darkness and light exposure are of substantially the same duration;
the duration is from about 2 hours to about 24 hours;
the duration is from about 6 hours to about 18 hours;
the duration is from about 10 hours to about 14 hours; and/or
where the duration is about 12 hours.

This aspect of the invention also relates to a substantially pure intact mushroom fruiting organism produced via the above process and any of its preferred embodiments.

In another of its aspects, the present invention relates to an isolated composition comprising mycelium of Psilocybe subtropicalis and a psychotropically active compound, wherein the psychotropically active compound is present in an amount of at least about 5 mg/g dried composition. Preferred embodiments of this isolated composition may include any one or a combination of any two or more of any of the following features:
the psychotropically active compound is present in an amount of at least about 8 mg/g dried mat composition;
the psychotropically active compound is present in an amount in the range of from about 8 to about 20 mg/g dried mat composition;
the psychotropically active compound is present in an amount in the range of from about 10 to about 20 mg/g dried mat composition;
the psychotropically active compound is present in an amount in the range of from about 12 to about 18 mg/g dried mat composition;
the psychotropically active compound is psilocybin;
the psychotropically active compound is psilocin; and/or
the psychotropically active compound is a mixture comprising psilocybin and psilocin.

The following is a brief glossary of terms.

As used throughout this specification, the term “about”, when used to describe a recited value, means within 5% of the recited value.

As used throughout this specification, the terms “comprising,” “having,” “including” and “containing,” and grammatical variations thereof, are inclusive or open-ended and do not exclude additional, un-recited elements and/or method steps. The term “consisting essentially of” when used herein in connection with a composition, use or method, denotes that additional elements, method steps or both additional elements and method steps may be present, but that these additions do not materially affect the manner in which the recited composition, method or use functions. The term “consisting of” when used herein in connection with a composition, use or method, excludes the presence of additional elements and/or method steps.

As used throughout this specification, the term “intact mushroom fruiting organism” is intended to mean a mushroom fruiting organism containing all major components. Typically, the major components of a mushroom fruiting organism produced according to this aspect of the invention are: all stages of the fruiting body (e.g., primordia, immature fruiting bodies, and mature fruiting bodies which may or may not be sporulating), the mycelium, spores, hyphae and volva.

As used throughout this specification, the term “substantially pure form” is intended to mean the mushroom fruiting organism is free of solid (i.e., non-liquid) growth media such as soil.

As used throughout this specification, the term “liquid growth medium” is intended to mean a non-solid medium of varying viscosity capable of growing a mushroom fruiting organism. Such a medium consists of a liquid growth (e.g., agar) medium with an added solidifying agent which includes but is not limited to agar.
Psilocybin fungi or psilocybin mushrooms—these are from a group of fungi that contain at least one psychoactive alkaloid, and generally contain psilocybin and psilocin. They may also contain other alkaloids such as baeocystin, norbaeocystin and norpsilocin.
Psilocybe mushrooms—these form a genus of gilled mushrooms in the family Hymenogastraceae. Most species contain the psychedelic alkaloids psilocybin and psilocin, and compounds such as baeocystin, norbaeocystin, and norpsilocin.
Psilocybin—this is a psychedelic prodrug produced by numerous species of mushrooms, collectively known as psilocybin mushrooms. Psilocybin is converted by the body to psilocin which has mind-altering effects.
Vessel—an enclosure in which the process for producing an intact mushroom fruiting organism is carried out. In a preferred embodiment, the vessel is configured to (i) permit air circulation between the interior and exterior of the vessel, and/or (ii) exclude ingress of microorganisms exterior to the vessel, and/or (iii) be made of a material which is radiation transparent (e.g., transparent to ambient radiation) such as glass.
Mycelial mat—the composition residing on or near the surface of the liquid growth medium after culturing fungal material (e.g., of Psilocybe subtropicalis). In a preferred embodiment, the mycelial mat is isolated (e.g., physically removed) from the liquid growth medium. In a preferred embodiment, the isolated mycelial mat is substantially dried, for example to reduce its water content.

In one of its aspects, the present invention relates to a process for the production of a psychotropically active compound comprising the step of culturing fungal material of Psilocybe subtropicalis. The terms “fungal material” and “mushrooms” are used interchangeably in the present specification

The fungal material used in the present processes can be any part of the mushroom fruiting organism. For example the fungal material used in the present processes can be one or more of: any stages of the fruiting body (e.g., primordia, immature fruiting bodies, and mature fruiting bodies which may or may not be sporulating), the mycelium (preferred), spores, hyphae and volva. In some embodiments, the raw psilocybin fungus parts used include only caps, or only stems, or only gills, or only hyphae or only mycelium or any mixture thereof. In still other cases, parts of the raw psilocybin fungus used are those that would normally be considered waste, in which valuable psychoactive compounds are found only in lower concentrations. The mushroom parts may be ground using a milling machine or pulverization device, for example.

The fungal material used in the aspect of the present process directed to production of a psychotropically active compound is preferably the mycelium, more preferably the mycelium of Psilocybe subtropicalis.

The fungal material used in the aspect of the present process directed to production of an intact mushroom fruiting organism is preferably inner cap tissue from a previously harvested mushroom fruiting body. Preferably, Psilocybe subtropicalis is used as the source of previously grown mushroom fruiting organism.

In a preferred embodiment, the yield of psychoactive tryptamines from mycelium grown as described can be comparable to what is seen in fruiting bodies despite a much shorter cultivation time.

In preferred embodiments, the uniform nature of liquid media (vs. solid substrate), controlled supplementation, as well as the use of mycelia (vs. mushrooms) fosters high levels of homogeneity and reproducibility.

The culturing/harvesting step in the present process is preferably conducted in liquid growth medium. In a preferred embodiment, the liquid growth medium is an aqueous liquid growth medium.

In a preferred embodiment, the media composition comprises: a yeast extract (preferred concentration of 3 g/L), a malt extract (preferred concentration of 3 g/L), optionally peptone (preferred concentration of 5 g/L), dextrose (preferred concentration of 10 g/L), tryptamine (preferred concentration of 1 g/L) and agar (preferred concentration of 10 g/L).

Embodiments of the present invention will be illustrated in the following Examples which should not be used to construe or limit the scope of the present invention.

### Example 1

5 mL of a well-homogenized Psilocybe subtropicalis suspension in 2% potato dextrose broth (i.e., the mother culture) was transferred to a bottle containing 100 mL containing sterilized yeast malt broth and 1.2 mM tryptamine. The mother culture is usually liquid nutrient media of potato dextrose broth comprising a pre-prepared mixture of 4.0 g/L potato infusion and 20.0 g/L dextrose inoculated with a 10 mm core of fully colonized agar containing mycelium of Psilocybe subtropicalis and allowed to grow for two weeks.

After 3 weeks of undisturbed growth (22-25 °C, 12/12 light/dark cycle), a dense mycelial mat was present at the top of the broth in all bottles.

The mycelial mats were harvested (isolated), and after complete drying, they were ground with a coffee grinder (Shardor, USA) and mortar and pestle to a fine powder. 50 mg was then extracted thrice with a mixture of 1 ml methanol-water (9:1, v/v). The extracts were vortexed for 2 min and then centrifuged at 3000 rpm at room temperature for 60 seconds. The combined supernatant was then centrifuged at 13000 rpm for 10 min at 22 °C and then subjected to HPLC analysis.

A HPLC 1290 Infinity assembly (Agilent Technologies, USA) with a Zorbax Eclipse XDB-C 18 column (4.6×150 mm, 3.5 μm) preceded by a Zorbax XDB-C18 guard column (4.6×12.5 mm, 5 μm) (Agilent Technologies, USA) at 30 °C was used for the chromatographic separation. Solvent A was 1% formic acid in acetonitrile while Solvent B was 10 mM ammonium acetate acidified to pH 4.6 with glacial acetic acid. The flow rate was 0.5 mL/min and the injection volume of the sample was 10 μL. The total run time of the separation was 12 min. The chromatographic method held the initial composition 10% Solvent A for 2 min increasing up to 35% Solvent A within 3 min, then further stepped to 100% Solvent A at 6 min and held for 0.5 min. It then followed a linear gradient to 95% Solvent B at 8 min and held for 2 min. It was then reduced down to 90% Solvent B at 11 min and held for 1 min. Chromatograms were recorded at λ=269 nm (4 nm) while the respective diode array detectors covered the wavelength range of λ=200-450 nm.

The above methodology was repeated for three additional psilocybin-producing fungal species: Psilocybe cubensis, Panaeolus cyanescens, and Gymnopilus sp.

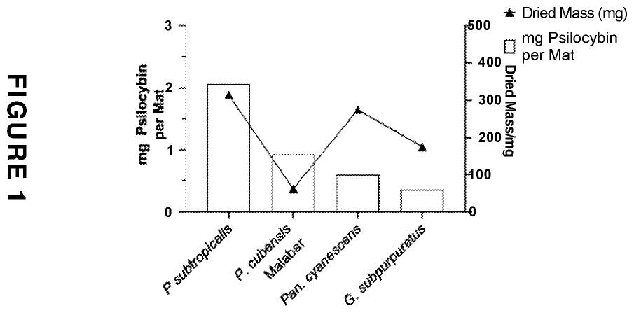

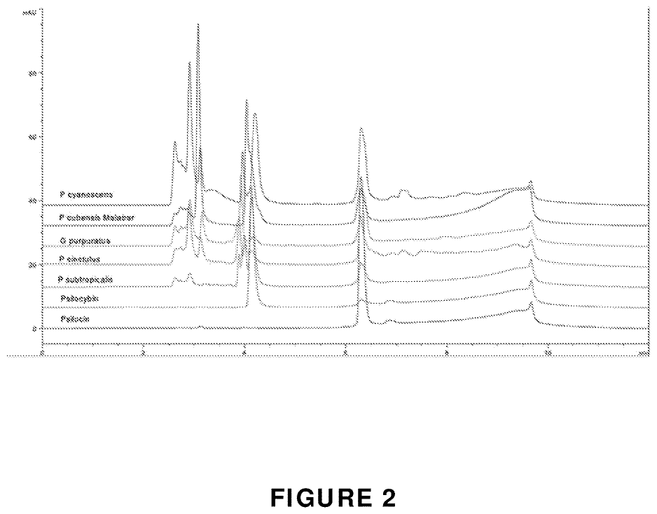

The results are shown in FIGS. 1 and 2.

As shown, the selection of Psilocybe subtropicalis as the source fungal material surprisingly resulted in significantly higher yields of psilocybin per mat and dried mass compared to when Psilocybe cubensis, Panaeolus cyanescens, and Gymnopilus sp. were chosen as the source fungal material. These improved results were seen after a harvest period of 3 weeks—i.e., obtaining heavier mats much more quickly than known harvesting processes which typically take 2-3 months.

These results demonstrate that the present process has the potential to significantly improve yields of the desired psychotropically active compound and to do so in significantly less time than is conventionally required to process a psilocybin-producing fungus.

### Example 2

A piece of cap tissue was taken from a mushroom fruiting body obtained from Psilocybe subtropicalis and transferred to a bottle containing 100 mL sterilized yeast malt broth and 1.2 mM tryptamine.

After 4 weeks of undisturbed growth (22-25 °C, 12/12 light/dark cycle), a dense mycelial mat was present at the top of the broth with fruiting bodies emerging from the mat.

The fruiting bodies were harvested, and after complete drying, they were ground with a coffee grinder (Shardor, USA) and mortar and pestle to a fine powder. 25 mg was then extracted thrice with a mixture of 1 ml methanol-water (9:1, v/v). The extracts were vortexed for 2 min and then centrifuged at 3000 rpm at room temperature for 60 seconds. The combined supernatant was then centrifuged at 13000 rpm for 10 min at 22 °C and then subjected to HPLC analysis.

A HPLC 1290 Infinity assembly (Agilent Technologies, USA) with a Zorbax Eclipse XDB-C18 column (4.6×150 mm, 3.5 μm) preceded by a Zorbax XDB-C18 guard column (4.6×12.5 mm, 5 μm) (Agilent Technologies, USA) at 30 °C was used for the chromatographic separation. Solvent A was 1% formic acid in Acetonitrile while Solvent B was 10 mM ammonium acetate acidified to pH 4.6 with glacial acetic acid. The flow rate was 0.5 mL/min and the injection volume of the sample was 10 μL. The total run time of the separation was 12 min. The chromatographic method held the initial composition of 10% Solvent A for 2 min increasing up to 35% Solvent A within 3 min, then further stepped to 100% Solvent A at 6 min and held for 0.5 min. It then followed a linear gradient to 95% Solvent B at 8 min and held for 2 min. It was then reduced down to 90% Solvent B at 11 min and held for 1 min. Chromatograms were recorded at λ=269 nm (4 nm) while the respective diode array detectors covered the wavelength range of λ=200-450 nm.

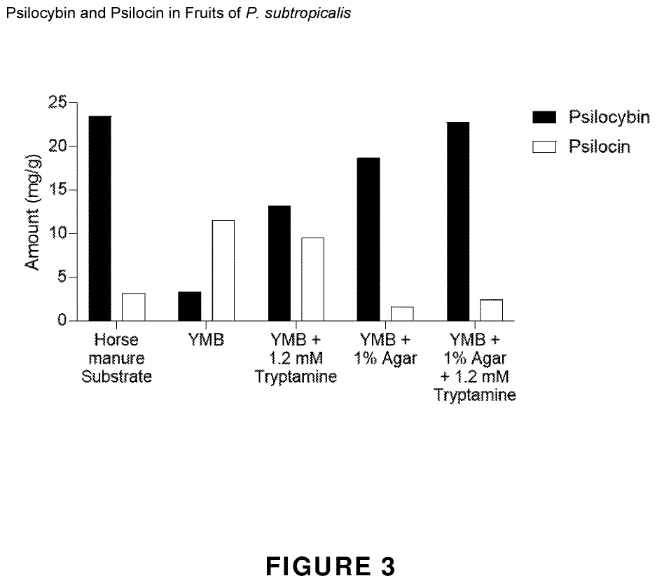

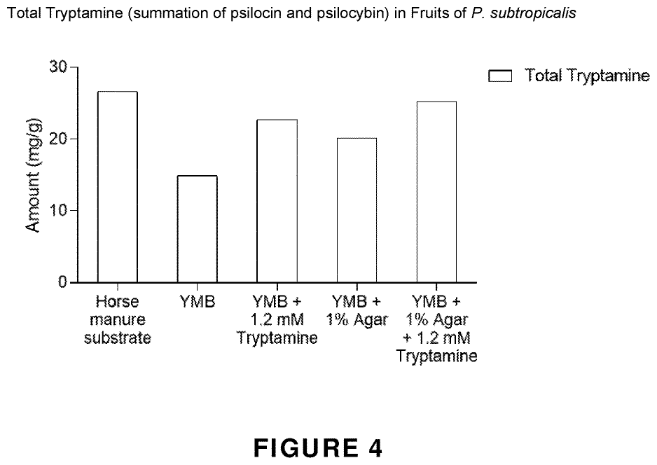

The results are shown in FIGS. 3 and 4.

As shown, the selection of the cap of Psilocybe subtropicalis as the source fungal material resulted in fruiting bodies with comparable yields of psilocybin, similar to when fruiting bodies are obtained from solid substrate. These results were seen after an incubation period of 4 weeks—i.e., much more quickly than the known harvesting process from solid substrate which typically takes 2-3 months. Moreover, such a process circumvents multiple of the normally requisite steps in the cultivation process (e.g., inoculation of grain and spawning to bulk substrate).

These results demonstrate that the present process has the potential to reliably produce sterile fruiting bodies with potent yields of the desired psychotropically active compounds and to do so in significantly less time and in less steps than is conventionally required to obtain fruiting bodies on solid substrate.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

1. A process for producing of a psychotropically active compound comprising the steps of: (a) culturing fungal material of Psilocybe subtropicalis in a growth medium to produce a composition comprising the psychotropically active compound, and (b) isolating the composition from the growth medium.

2. The process defined in claim 1, wherein the psychotropically active compound is selected from a group consisting of psilocybin, psilocin and a mixture thereof.

3. (canceled)

4. (canceled)

5. The process defined in claim 1, wherein the growth medium further comprises tryptamine.

6. The process defined in claim 5, wherein tryptamine at a concentration of greater than about 0.3 mM.

7. (canceled)

8. The process defined in claim 5, wherein tryptamine is present at a concentration in the range of from about 0.6 mM to about 1.5 mM.

9. (canceled)

10. The process defined in claim 1, wherein the step of culturing is conducted at a temperature in the range of from about 15 °C to about 30 °C.

11. (canceled)

12. (canceled)

13. (canceled)

14. (canceled)

15. (canceled)

16. (canceled)

17. The process defined in claim 1, wherein the step of culturing is conducted in cycles of darkness and light exposure.

18. The process defined in claim 17, wherein the cycles of darkness and light exposure are of substantially the same duration.

19. The process defined in claim 18, wherein the duration is from about 2 hours to about 24 hours.

20. (canceled)

21. (canceled)

22. (canceled)

23. (canceled)

24. (canceled)

25. The process defined in claim 1, wherein the step of culturing is conducted for a period of from 1 week to 12 weeks.

26. (canceled)

27. (canceled)

28. The process defined in claim 1, wherein the fungal material is the mycelia of Psilocybe subtropicalis and the composition produced in Step (b) is a mycelial mat.

29. An isolated and dried mycelial mat produced according to the process defined in claim 28.

30. A mycelial mat produced by culturing mycelia of Psilocybe subtropicalis in a growth medium.

31. A liquid growth medium composition comprising: a yeast extract, a malt extract, dextrose, tryptamine and, optionally, agar.

32. The growth medium composition defined in claim 31, wherein the yeast extract is present at a concentration in the range of from about 1 g/L to 5 g/L.

33. (canceled)

34. (canceled)

35. The growth medium composition defined in claim 31, wherein the malt extract is present at a concentration in the range of from about 1 g/L to 5 g/L.

36. (canceled)

37. (canceled)

38. (canceled)

39. (canceled)

40. (canceled)

41. (canceled)

42. The growth medium composition defined in claim 31, wherein dextrose is present at a concentration in the range of from about 8 g/L to 12 g/L.

43. (canceled)

44. (canceled)

45. The growth medium composition defined in claim 31, wherein tryptamine is present at a concentration in the range of from about 0.3 mM to about 2.0 mM.

46. (canceled)

47. (canceled)

48. The growth medium composition defined in claim 31, wherein agar is present at a concentration in the range of from about 0 g/L to 30 g/L.

49. The growth medium composition defined in claim 31, wherein agar is present at a concentration in the range of about 3 g/L to about 15 g/L,

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)
